(19) United States
(12) Patent Application Publication
(10) Pub. No.: US 2008/0118446 A1
(43) Pub. Date: May 22, 2008

(54) Smile shower

(76) Inventor: Jennifer Jablow, New York, NY (US)

Correspondence Address: WEISS & WEISS, Suite 251, 300 Old Country Road, Mineola, NY, 11501, US

(21) Appl. No.: 11/985,945

(22) Filed: Nov. 19, 2007

Related U.S. Application Data
(60) Provisional application No. 60/859,617, filed on Nov. 17, 2006.

(51) Int. Cl. A61K 8/34; A61K 8/00; A61K 8/21; A61Q 11/00; A61K 8/97; A61K 8/22

(52) U.S. Cl. 424/52; 424/49; 424/53; 424/58

(57) ABSTRACT

The present invention is a spray product used in the oral cavity. The present invention does not require brushing and it can be used outside the bathroom as opposed to conventional “toothpaste products”. The product of the present invention can be used to promote whitening, stain removal, anti caries, anti plaque, anti sensitivity, anti halitosis, anti xerostomia components, as well as promoting a healthy oral environment by incorporating natural antioxidants and creating a ph that allows for remineralization and adequate saliva flow. By addressing the oral health issues, overall body health will in turn be addressed. The beauty component is addressed by the whitening and stain removing/preventing ingredients.

### RELATED APPLICATIONS

This application claims priority to provisional patent application Ser. No. 60/859,617 filed Nov. 17, 2006.

### FIELD OF THE INVENTION

The present invention relates to a spray product that functions as a toothpaste, mouthwash, breath freshener and whitener all in one used in the oral cavity. The present invention does not require brushing and it can be used outside the bathroom as opposed to conventional “toothpaste products”. If the consumer chooses to use a toothbrush, the product will foam like a tube toothpaste. The product of the present invention can be used to promote whitening, stain removal and prevention, anti caries, anti plaque, anti sensitivity, anti halitosis, anti xerostomia components, as well as promoting a healthy oral environment by incorporating natural antioxidants and creating a pH that allows for remineralization and adequate saliva flow. By addressing the oral health issues, overall body health will in turn be addressed. The beauty component is addressed by the whitening and stain removing/preventing ingredients.

### BACKGROUND OF THE INVENTION

As the consumer becomes more educated about overall health and beauty, there are more oral care products being produced that are multi functional. Many of the products are toothpaste and mouthwashes i.e. Crest Pro Health (Proctor & Gamble), that are used in the traditional sense of brushing and rinsing. The product of the present invention allows the consumer to use it anytime without the need for brushing. Some “multi-care” products address the link between gum inflammation, plaque, and periodontal disease to health. They are also combined with whitening and anti caries agents. There is no oral product that is portable; “on the go” that addresses 1. chronic oral inflammatory conditions, 2. decay, 3. xerostomia, 4 halitosis, 5. Beauty through whitening and stain prevention/removal, 6. repair and remineralization, and 7. antioxidants

It has been proven through many documented studies that chronic inflammation even low grade, has a fundamental role in every disease and in speeding up the aging process. Many of the systemic problems Americans suffer from are linked to this destructive inflammatory process. 4 of 5 Americans have some form of periodontal condition (AAP, 2005). The inflammatory cell cascade from a chronic low level gingivitis can signal a domino effect in the rest of the body. People with periodontal disease conditions have proven increased risks of heart disease, stroke, Alzheimer's, respiratory disease and premature births. Scientifically, the inflammatory process can be simplified this way: the inflammatory cell (reacting to dental plaque), signal the pro-inflammatory cytokines (Interleukin-6), which in turn signals the enzyme collagenase. This enzyme is responsible for the destruction of collagen: the building block of bones, gum tissue, blood vessels, teeth, etc. By controlling the bacteria and the inflammatory process in the oral cavity, we can promote better overall systemic health.

Antioxidants are antidotes to help control inflammation. By incorporating them into oral care products through plant and fruit extracts, it may help control the propagation of collagen breakdown, inflammation, and premature aging in the mouth and body. By oral administration of these antioxidants, there will be absorption into the oral tissues and the whole body system. Low grade inflammatory disorders if not controlled can have chronic systemic effects. Adding natural anti inflammatories to multi care oral products can help in the prevention of more serious inflammatory diseases such as heart disease, Alzheimer's and osteoarthritis. By making it convenient for the consumer to use in a mouth spray and liquid spray paste, that also freshens, whitens and moistens the mouth can increase incorporation of anti oxidants (natural anti inflammatories) into our daily regimen.

### SUMMARY OF THE INVENTION

The present invention relates to a spray product comprising: Xylitol, a whitening agent, a stain remover and/or prevention agents, one or more antioxidants, and fluoride. It is an object of the present invention for the spray product to further comprise breath freshening agents. It is an object of the present invention for the stain remover and or prevention agents to comprise PVP (Plasdone from ISP corp, NJ). It is an object of the present invention for the whitening agent to comprise Hydrogen peroxide. It is an object of the present invention for the antioxidants to have dental applications and overall health benefits.

It is an object of the present invention for the spray to further comprise: sodium bicarbonate (baking soda). It is an object of the present invention for the PVP (PLASDONE, ISP corp., NJ/food grade) to comprises approximately 0.01% to 40% of the spray product. It is an object of the present invention for the spray product to be comprised of hydrogen peroxide of approximately 0.01% to 5%. It is an object of the present invention for the spray to further comprise: spearmint, peppermint oil or both or any mint combination with a fruit flavor, vanilla or chocolate. It is an object of the present invention for the spray to comprise aloe vera. It is an object of the present invention for the spray to comprise: glycerin. It is an object of the present invention for the spray to comprise purified water. It is an object of the present invention for the spray to comprise a source of calcium, such as calcium carbonate, or calcium phosphate. It is an object of the present invention for the spray to comprise other inactive ingredients including sorbitol, poloxamer 407, cocamidopropyl betaine, polysorbate 80, parabens or sodium benzoate, sodium hydroxide, and sucralose. It is an object of the present invention for the fluoride concentration (sodium fluoride USP) to be approximately in the range from 0.01 to 0.5%. It is an object of the present invention for the Xylitol to be in the range from approximately 0.01 to 15%.

It is an object of the present invention for the antioxidant source to comprise one or a combination of green tea, cranberry extract and/or pomegranite.

It is an object of the present invention to provide a portable spray that gives a person long lasting fresh breath.

It is an object of the present invention to provide a portable spray that whitens teeth. It is an object of the present invention to provide a portable spray that protects against cavities (Anti-Caries).

It is an object of the present invention to provide a portable spray that protects against gum disease. It is an object of the present invention to provide a portable spray that prevents inflammation.

It is an object of the present invention to provide a portable spray that provides an antioxidant and vitamins for the mouth and whole body.

It is an object of the present invention to provide a portable spray that assists with Anti Aging by preventing collagen breakdown from inflammation. It is an object of the present invention to provide a portable spray that provides a moisturizing agent, anti-xerostomia.

It is an object of the present invention to provide a portable spray that provides anti sensitivity. It is an object of the present invention to provide a portable spray that repairs and remineralizes a person's teeth.

It is an object of the present invention to provide a portable spray that is alcohol free. It is an object of the present invention to provide a portable spray that promotes a healthy mouth environment and controls pH.

It is an object of the present invention to provide a portable spray that is safe to use on crowns, bonding and veneers.

It is an object of the present invention to provide a portable spray that is portable, and can be used anywhere, and does not require brushing. If a toothbrush is used, the product will foam like a traditional tube paste.

### DETAILED DESCRIPTION

The present invention relates to a spray product comprising: Xylitol, a whitening agent, such as hydrogen peroxide, an antioxidant source, fluoride, and a stain removal agent, PLASDONE (PVP). In a further embodiment, the spray further comprises: glycerin, spearmint, peppermint oil or both, aloe vera, glycerin, purified water, and a source of calcium, alone or in combination.

Xylitol has reduces cavities and remineralizes tooth structure. Xylitol increases salivary flow which helps remineralize teeth. Xylitol helps with nasal problems.

Cranberry extract prevents bacteria from adhering to tooth structure. It also has overall health benefits from its antioxidant properties that fight inflammation.

In a preferred embodiment, the whitening agent comprises a small amount of hydrogen peroxide or calcium peroxide or both.

PVP works with the peroxide to remove stains and therefore “whiten” teeth. Aloe Vera has a soothing and repairing effect.

Glycerin, or a moisturizing agent (vegetable source) can also be added to the spray product.

In an embodiment, the antioxidant sources may consist of green tea extract known to have the highest amount of polyphenols of the teas, EGCG, which helps inhibit bacteria (strep mutants) from adhering to tooth and helps in collagen repair. Cranberry extract provides anti caries which inhibit strep mutants which adhere to tooth and an antioxidant.

In an embodiment, the spray product further comprises purified water.

In a preferred embodiment, a preservative can be added if necessary such as parabens or sodium benzoate.

In an embodiment, the spray product contains fluoride, in a concentration range from approximately 0.01% to 0.5%.

The pH of the product ranges from 5 to 7.5.

In an embodiment, the spray product may contain a source of calcium preferably, calcium carbonate, or calcium phosphate.

The spray product can be dispensed from a 0.25 fl oz bottle to 4 fl oz bottle.

The product can be used without a brush by simply spraying into the mouth, swishing it around for up to 2 minutes and expectorating. The product can be used several times a day. A toothbrush can be used which will foam the liquid like a traditional paste.

1. A spray product for promoting a healthy oral cavity comprising:
Xylitol;
a whitening agent;
an antioxidant source;
and a stain remover/prevention agent.

2. The spray product of claim 1 wherein said stain remover/prevention agent comprises PVP (PLASDONE).

3. The spray product of claim 1 wherein said whitening agent comprises Hydrogen peroxide.

4. The spray product of claim 1 further comprising fluoride.

5. The spray product of claim 1 further comprising: spearmint, peppermint oil or a combination with fruit, vanilla or chocolate.

6. The spray product of claim 1 further comprising: aloe vera.

7. The spray product of claim 1 further comprising: glycerin.

8. The spray product of claim 1 further comprising: purified water.

9. The spray product of claim 1 further comprising: a source of calcium, such as calcium carbonate, or calcium phosphate.

10. The spray product of claim 1 wherein said antioxidant source comprises green tea, cranberry extract and/or pomegranite.

11. The spray product of claim 1 wherein said whitening agent comprises a small amount of hydrogen peroxide or calcium peroxide or both.

12. The spray product of claim 1 wherein said spray product further comprises:
breath freshening agents.

13. The spray product of claim 1 wherein said spray product further comprises:
sodium bicarbonate (baking soda).

14. The spray product of claim 2 wherein said PVP comprises approximately 0.01% to 40% of said spray product.

15. The spray product of claim 3 wherein said spray product comprises hydrogen peroxide of approximately 0.01% to 5% of said spray product.

16. The spray product of claim 4 wherein said fluoride concentration is approximately 0.01 to 0.5% of said spray product.

17. The spray product of claim 1 wherein said Xylitol is in range from approximately 0.01 to 15% of spray product.

18. The spray product of claim 1 wherein said spray is alcohol free.

19. The spray product of claim 1 wherein said spray product has a pH of approximately 5 to 7.5.

20. The spray product of claim 1 further comprising a preservative such as parabens or sodium benzoate.
